SEQUENCE LISTING (41 sequences)

SEQ ID NO: 1 - DNA, 990 bp, Salmonella typhimurium
gtgactctggtcgacgaacttaaataatgcctgcctcaccctcttttcttcagaaagagggtgactatttgtctggtttattaactgtttatccccaaagcaccataatcaacgctagactgttcttattgttaacacaagggagaagagatgatgcgcgtactggttgtagaggataatgcattattacgccaccacctgaaggttcagctccaggattcaggtcaccaggtcgatgccgcagaagatgccagggaagctgattactaccttaatgaacaccttccggatatcgctattgtcgatttaggtctgccggatgaagacggcctttccttaatacgccgctggcgcagcagtgatgtttcactgccggttctggtgttaaccgcgcgcgaaggctggcaggataaagtcgaggttctcagctccggggccgatgactacgtgacgaagccattccacatcgaagaggtaatggcgcgtatgcaggcgttaatgcgccgtaatagcggtctggcctcccaggtgatcaacatcccgccgttccaggtggatctctcacgccgggaattatccgtcaatgaagaggtcatcaaactcacggcgttcgaatacaccattatggaaacgcttatccgtaacaacggtaaagtggtcagcaaagattcgctgatgcttcagctgtatccggatgcggaactgcgggaaagtcataccattgatgttctcatggggcgtctgcggaaaaaaatacaggcccagtatccgcacgatgtcattaccaccgtacgcggacaaggatatctttttgaattgcgctaatgaataaatttgctcgccattttctgcgtgtcgctgcgggttcgttttttgctggcgacagccggcgtcgtgctggtgctttctttggcatatggcatagtggcgctggtcggctatagcgtaagttttgataaaaccacctttcgtttgctgcgcggcgaaagc

SEQ ID NO: 2 - DNA, 160 bp, Bacillus haldurans
gtgacgttattgcaatttaatcttgaacagtcaggctacgaggtcgtgacagcaatggatggagcttctgggctacaactagctaagacgcaaacgttcgatcttattattttagacctcatgttacctgaaatggatggactcgatgtatgtaaacaac

SEQ ID NO: 3 - DNA, 160 bp, Bacillus subtilis
gttactcttttacagtacaatttggaacggtcaggctatgatgtcattaccgcctcggatggggaagaagcactcaaaaaagcggaaacagagaaacctgatttgattgtgcttgatgtgatgcttccaaaattggacggaatcgaagtatgcaagcagc

SEQ ID NO: 4 - DNA, 160 bp, Clostridium acetobutylicum
tcaaatttgataaagttaaatttaaatatggcgggatatataagtgaagctgtgtataatggtgaagctgcactggacttaattgaaggtagaaattttgatttaatacttttagacataatgctgcctaaaatagatggttttagtctatttcaaaaaa

SEQ ID NO: 5 - DNA, 160 bp, Escherichia coli
cgtcaccaccttaaagttcagattcaggatgctggtcatcaggtcgatgacgcagaagatgccaaagaagccgattattatctcaatgaacatataccggatattgcgattgtcgatctcggattgccagacgaggacggtctgtcactgattcgccgct

SEQ ID NO: 6 - DNA, 160 bp, Escherichia coli; identical to SEQ ID NO: 5

SEQ ID NO: 7 - DNA, 160 bp, Escherichia coli; identical to SEQ ID NO: 5

SEQ ID NO: 8 - DNA, 160 bp, Escherichia coli
cgtcaccaccttaaagttcagattcaggatgctggtcatcaggtcgatgatgcagaagatgccaaagaagccgattattatctcaatgaacatttaccggatattgcgattgtcgatctcggattgccagacgaggacggtctgtcactgatttgccgct

SEQ ID NO: 9 - DNA, 160 bp, Listeria innocua
gttaccttgttgcaatttaatattgaaaaagctgggtttgatgtagtcacagctgaagatggtagaactgggtacgaacttgctctatcggaaaaaccagatttaattgtacttgatttaatgcttcctgaaatggacggaattgaagtaacgaaaaaac

SEQ ID NO: 10 - DNA, 160 bp, Listeria innocua; identical to SEQ ID NO: 9

SEQ ID NO: 11 - DNA, 160 bp, Listeria monocytogenes; identical to SEQ ID NO: 9

SEQ ID NO: 12 - DNA, 160 bp, Listeria monocytogenes
gttaccttgctacaatttaatattgaaaaagcaggatttgaagtggtgacagctgaagatggtagaactgggtatgagctcgctttgtccgaaaagccagatttaattgtgcttgatttaatgcttcctgagatggacggaatcgaagtaacaaaaaaac

SEQ ID NO: 13 - DNA, 160 bp, Mycobacterium leprae
gtcgaaccgctctaggtgacatcaaattccagggcttttaggtccaggctgtgtttaaaggagccgcggcagctggactaggctcgtagtgctcggccggacgcggtgatcttggacgtggtgatgccggggatggacggtttcggggtgctgcgctggc

SEQ ID NO: 14 - DNA, 160 bp, Mycobacterium tuberculosis
gttgaactgctgtcggtgagcctcaagttccagggctttgaagtctacaccgcgaccaacggggcacaggcgctggatcgggcccgggaaacccggccggacgcggtgatcctcgatgtgatgatgcccgggatggacggctttggggtgctgcgccggc

SEQ ID NO: 15 - DNA, 160 bp, Pseudomonas aeruginosa
cgccaccacctctatacccgcctgggtgaacaggggcacgtggtggacgcggtaccggatgccgaggaagccctctaccgggtcagcgaataccaccacgacctggcggtgatcgacctcggcctgccgggcatgagcggcctggacctgatccgcgagc

SEQ ID NO: 16 - DNA, 160 bp, Salmonella typhimurium
cgccaccacctgaaggttcagctccaggattcaggtcaccaggtcgatgccgcagaagatgccagggaagctgattactaccttaatgaacaccttccggatatcgctattgtcgatttaggtctgccggatgaagacggcctttccttaatacgccgct

SEQ ID NO: 17 - DNA, 160 bp, Salmonella typhimurium; identical to SEQ ID NO: 16

SEQ ID NO: 18 - DNA, 160 bp, Salmonella enterica; identical to SEQ ID NO: 16

SEQ ID NO: 19 - DNA, 160 bp, Salmonella enterica; identical to SEQ ID NO: 16

SEQ ID NO: 20 - DNA, 160 bp, Salmonella typhimurium; identical to SEQ ID NO: 16

SEQ ID NO: 21 - DNA, 160 bp, Salmonella typhimurium; identical to SEQ ID NO: 16

SEQ ID NO: 22 - DNA, 160 bp, Salmonella typhimurium; identical to SEQ ID NO: 16

SEQ ID NO: 23 - DNA, 160 bp, Staphylococcus aureus
gtaacattacttaaatataacttagaaacagctggttatgaagttgttgtcgcatttgatggtgatgaggctttagaaaaggtagaaagtgaacagccagatttaattattttagatgttatgctacctaaaaaagatggcattgacgtatgtaagactg

SEQ ID NO: 24 - DNA, 160 bp, Staphylococcus aureus; identical to SEQ ID NO: 23

SEQ ID NO: 25 - DNA, 160 bp, Streptococcus pneumoniae
ctgaaattgcttgactaccatttaagtaaggaaggcttttctactcaattggtgacaaatggacggaaggccttagctttggcagaaacagaaccctttgattttatcttgcttgatatcatgttaccacaattagatggcatggaagtttgtaagcggc

SEQ ID NO: 26 - DNA, 160 bp, Yersinia pseudotuberculosis
cgtcaccatctgacagtgcaaatgcgtgaaatgggccatcaggttgatgccgcggaagatgctaaagaagcagactatttcttacaagagcatgcccccgacattgctattatcgatcttggtttgcccggtgaagacgggttaagccttatccgtcgct

SEQ ID NO: 27 - DNA, 160 bp, Yersinia pestis; identical to SEQ ID NO: 26

SEQ ID NO: 28 - DNA, 160 bp, Yersinia pestis; identical to SEQ ID NO: 26

SEQ ID NO: 29 - DNA, 160 bp, Yersinia pseudotuberculosis; identical to SEQ ID NO: 26

SEQ ID NO: 30 - DNA, 137 bp, Salmonella
ctccaggattcaggtcaccaggtcgatgccgcagaagatgccagggaagctgattactaccttaatgaacaccttccggatatcgctattgtcgatttaggtctgccggatgaagacggcctttccttaatacgccg

SEQ ID NO: 31 - DNA, 25 bp, Salmonella
tattgtcgatttaggtctgccggat

SEQ ID NO: 32 - DNA, 18 bp, Artificial
  PCR Primer
ctccaggattcaggtcac

SEQ ID NO: 33 - DNA, 18 bp, Artificial
  PCR primer
cggcgtattaaggaaagg

SEQ ID NO: 34 - DNA, 37 bp, Artificial
  Molecular Beacon
cgtcgctattgtcgatttaggtctgccggatgcgacg

SEQ ID NO: 35 - DNA, 25 bp, Artificial
  Molecular beacon loop
tattgtcgatttaggtctgccggat

SEQ ID NO: 36 - DNA, 37 bp, Artificial
  Molecular beacon
cgtcgcatccggcagacctaaatcgacaatagcgacg

SEQ ID NO: 37 - DNA, 25 bp, Artificial
  Molecular beacon loop
atccggcagacctaaatcgacaata

SEQ ID NO: 38 - DNA, 36 bp, Artificial Sequence
  Molecular beacon
cgacgctgaacaccttccggatatcgctatgcgtcg

SEQ ID NO: 39 - DNA, 24 bp, Artificial Sequence
  Molecular beacon loop
tgaacaccttccggatatcgctat

SEQ ID NO: 40 - DNA, 36 bp, Artificial Sequence
  Molecular beacon
cgacgcatagcgatatccggaaggtgttcagcgtcg

SEQ ID NO: 41 - DNA, 24 bp, Artificial Sequence
  Molecular beacon loop
atagcgatatccggaaggtgttca

(19) United States
(12) Patent Application Publication
(10) Pub. No.: US 2007/0134659 A1
(43) Pub. Date: Jun. 14, 2007

(54) Polynucleotides for the detection of salmonella species

(75) Inventors: Daniel Plante, Quebec (CA); Eliane Ubalijoro, Quebec (CA)

Correspondence Address: STERNE, KESSLER, GOLDSTEIN & FOX P.L.L.C., 1100 NEW YORK AVENUE, N.W., WASHINGTON, DC, 20005, US

(73) Assignee: Warnex Reasearch, Inc., Laval, Quebec (CA)

(21) Appl. No.: 10/553,706

(22) PCT Filed: Apr. 19, 2004
(86) PCT No.: PCT/CA04/00576

§ 371 Date: Aug. 16, 2006

Related U.S. Application Data
(60) Provisional application No. 60/463,741, filed on Apr. 18, 2003.

(51) Int. Cl. C12Q 1/68; C07H 21/04

(52) U.S. Cl. 435/6; 536/24.1; 536/25.32

(57) ABSTRACT

Polynucleotide primers and probes for the amplification and detection of Salmonella species in samples are provided. The primers and probes can be used in real time diagnostic assays for rapid detection of one or more Salmonella species in a variety of situations. Kits comprising the primers and probes are also provided

### FIELD OF THE INVENTION

The present invention pertains to the field of detection of microbial contaminants, and in particular to the detection of contamination by Salmonella species.

### BACKGROUND OF THE INVENTION

The genus Salmonella is composed of seven species and Salmonella strains are responsible for a large number of reported cases of food poisoning throughout the world. This bacterium is commonly associated with contamination of foods such as milk, milk products, seafood, poultry and meat. Within 12 to 36 hours of ingestion, individuals infected by the pathogen may develop symptoms ranging from diarrhoea, stomach cramps, and in more severe cases vomiting and fever. In order to prevent Salmonella infections, methods of detection can be utilized that identify the presence of the bacteria in food, prior to consumer availability and consumption. However, due to relatively quick rates of food spoilage, many detection techniques, which require long time periods, are not time and cost effective. For example, a number of detection technologies require the culturing of bacterial samples for time periods of up to eight days. However, in that time, the product being tested must be placed in circulation for purchase and consumption. Therefore, a system that can rapidly identify the presence of Salmonella in food samples is desirable.

A variety of methods are described in the art for the detection of bacterial contaminants. One of these methods is the amplification of specific nucleotide sequences using specific primers in a PCR assay. Upon completion of the amplification of a target sequence, the presence of an amplicon is detected using agarose gel electrophoresis. For example, U.S. Pat. No. 5,795,717 describes PCR amplification of a portion of the araC gene, which is believed to be common to all Salmonella species, and detecting the amplified region by agarose gel electrophoresis. This method of detection, while being more rapid than traditional methods requiring culturing bacterial samples, is still relatively time consuming and subject to post-PCR contamination during the running of the agarose gel.

An additional technology utilized for detection of bacterial contamination, is nucleic acid hybridization. In such detection methodologies, the target sequence of interest is amplified and then hybridized to an oligonucleotide probe which possesses a complementary nucleic acid sequence to that of the target molecule. The probe can be modified so that detection of the hybridization product may occur, for example, the probe can be labelled with a radioisotope or fluorescent moiety.

The general use of Salmonella nucleic acid sequences for detection of the bacterium has been described. For example, U.S. Pat. No. 5,486,454 describes a nucleic acid probe derived from the nucleotide sequences of a gene encoding Type I fimbriae protein that is useful for detecting Salmonella spp. in diarrhoea specimens. In another example, International Patent Application No. PCT/IB94/00205 (WO 94/25597) describes isolated nucleic acid probes and primers complementary to or derived from one or more of a number of the Salmonella sef genes, agfA, tctA, tctB, or tctC genes that are useful for the detection of Salmonella spp. and/or other enteropathogenic bacteria. European Patent Application No. EP 0 721 989, describes the use of oligonucleotides based on the iagA and iagB genes for the detection of Salmonella and U.S. Pat. No. 6,165,721, describes oligonucleotide primers and probes targeting spaO and spaQ genes, that are useful for amplification and detection of a variety of Salmonella strains and serotypes. International Patent Application No. PCT/GB94/01316 (WO 95/00664) describes the detection of bacteria of the Salmonella genus using nucleic acid molecules as probes or primers in DNA-based detection systems, however, a number of representative Salmonella subspecies (e.g. subspecies arizonae) could not be not detected with these systems. International Patent Application No. PCT/EP98/05129 (WO 99/07886) describes an improved method that is based on identifying phylogenetically conserved base sequences within the target sequence described in WO 95/00664. The preparation and use of probes that are capable of hybridizing to a unique region of rRNA and detecting most, but not all, Salmonella species is described in U.S. Pat. Nos. 5,714,321 and 5,147,778.

A particularly useful modification of the above hybridization technology provides for the concurrent amplification and detection of the target sequence (i.e. in “real time”) through the use of specially adapted oligonucleotide probes. Examples of such probes include molecular beacon probes (Tyagi et al., (1996) Nature Biotechnol. 14:303-308), TaqMan® probes (U.S. Pat. Nos. 5,691,146 and 5,876,930) and Scorpion probes (Whitcombe et al., (1999) Nature Biotechnol. 17:804-807). For example, International Patent Application No. PCT/US02/21181 (WO 03/000935), describes a method for detecting a Salmonella species by amplifying a genomic nucleotide sequence of the sipB-sipC gene region of the Salmonella genome by real-time PCR and detecting the amplification product by FRET using a pair of labelled polynucleotides. In another example, International Patent Application PCT/US01/25231 (WO 02/14555) describes detection of Salmonella using single-labelled oligonucleotide probes that target the Salmonella spaQ gene in real-time.

Molecular beacons represent a powerful tool for the rapid detection of specific nucleotide sequences and are capable of detecting the presence of a complementary nucleotide sequence even in homogenous solutions. Molecular beacons can be described as hairpin stem-and-loop oligonucleotide sequences, in which the loop portion of the molecule represents a probe sequence, which is complementary to a predetermined sequence in a target nucleotide. One arm of the beacon sequence is attached to a fluorescent moiety, while the other arm of the beacon is attached to a non-fluorescent quencher. The stem portion of the stem-and-loop sequence holds the two arms of the beacon in close proximity. Under these circumstances, the fluorescent moiety is quenched. When the beacon encounters a nucleic acid sequence complementary to its probe sequence, the probe hybridizes to the nucleic acid sequence, forming a stable complex and, as a result, the arms of the probe are separated and the fluorophore emits light. Thus, the emission of light is indicative of the presence of the specific nucleic acid sequence. Individual molecular beacons are highly specific for the DNA sequences they are complementary to. The use of molecular beacons for the detection of Salmonella has been previously described. For example, International Patent Application PCT/IJS99/10940 (WO 99/63112) describes a method of detecting microbial contaminants in foodstuffs utilizing probes and primers that target universal or specific microbial nucleic acid sequences (e.g. the lamB gene for detection of E. coli, Salmonella and Shigella; and the DNA replication origin for detection of Salmonella).

PhoP is a DNA-binding partner of the two-component response regulatory system phoP-phoQ. This system is activated after the bacteria enter host cells and regulates transcription of diverse bacterial genes including at least 40 virulence factors. When PhoP is phosphorylated, it becomes active, functioning as a transcriptional regulator of PhoP-activated genes and PhoP-repressed genes in turn controlling the expression of a number of genes important for macrophage survival. It has been demonstrated that phoP expression affects host cell antigen processing and presentation. PhoP also induces genes involved in magnesium transport and has been shown to play a role in bacterial resistance to bile [Beuzon C R, et al. (2001). Infection and Immunology 69:7254-61; Detweiler C S et al. (2001). PNAS (USA) 98:5850-5; Heithoff D M, et al. (1997) PNAS (USA) 94:934-9].

Identification of genes specifically induced during microbial infection has been described in U.S. Pat. Nos. 6,365,401 and 6,548,246. These patents describe the use of In vivo Expression Technology (IVET), utilising fragments of genomic DNA from S. typhimurium to identify genes that are involved in Salmonella virulence. The methodology was intended to identify unknown genes involved in virulence in addition to virulence genes found in other pathogens, but not previously known to exist in Salmonella spp. As expected, the coding sequences of induced genes known to be implicated in Salmonella virulence, such as the phoPQ genes, were also detected.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide polynucleotides for the detection of Salmonella. In accordance with one aspect of the present invention, there is provided a combination of polynucleotides for amplification and detection of a portion of a Salmonella phoP gene, said portion being less than about 500 nucleotides in length and comprising at least 60 consecutive nucleotides of the sequence set forth in SEQ ID NO:30, said combination comprising: a first polynucleotide primer comprising at least 7 nucleotides of the sequence as set forth in SEQ ID NO:1; a second polynucleotide primer comprising at least 7 nucleotides of a sequence complementary to SEQ ID NO:1; and a polynucleotide probe comprising at least 7 consecutive nucleotides of the sequence as set forth in SEQ ID NO:30, or the complement thereof

In accordance with another aspect of the invention, there is provided a pair of polynucleotide primers for amplification of a portion of an Salmonella phoP gene, said portion being less than about 500 nucleotides in length and comprising at least 60 consecutive nucleotides of the sequence set forth in SEQ ID NO:30, said pair of polynucleotide primers comprising: a first polynucleotide primer comprising at least 7 nucleotides of the sequence as set forth in SEQ ID NO:1; and a second polynucleotide primer comprising at least 7 nucleotides of a sequence complementary to SEQ ID NO:1.

In accordance with another aspect of the invention, there is provided a method of detecting one or more Salmonella species in a sample, said method comprising: contacting a test sample suspected of containing, or known to contain, a Salmonella target nucleotide sequence with a combination of polynucleotides of the invention under conditions that permit amplification and detection of said target sequence, and detecting any amplified target sequence, wherein detection of an amplified target sequence indicates the presence of one or more Salmonella species in the sample.

In accordance with another aspect of the invention, there is provided a kit for the detection of one or more Salmonella species in a sample, said kit comprising: a first polynucleotide primer comprising at least 7 nucleotides of the sequence as set forth in SEQ ID NO:1; a second polynucleotide primer comprising at least 7 nucleotides of a sequence complementary to SEQ ID NO:1; and a polynucleotide probe comprising at least 7 consecutive nucleotides of the sequence as set forth in SEQ ID NO:30, or the complement thereof.

In accordance with another aspect of the invention, there is provided an isolated Salmonella specific polynucleotide having the sequence as set forth in SEQ ID NO:30, or the complement thereof.

In accordance with another aspect of the invention, there is provided a polynucleotide primer of between 7 and 100 nucleotides in length for the amplification of a portion of a Salmonella phoP gene, said polynucleotide comprising at least 7 consecutive nucleotides of the sequence as set forth in SEQ ID NO:30, or the complement thereof.

In accordance with another aspect of the invention, there is provided a polynucleotide probe of between 7 and 100 nucleotides in length for detection of Salmonella, said polynucleotide comprising at least 7 consecutive nucleotides of the sequence as set forth in SEQ ID NO:30, or the complement thereof.

### BRIEF DESCRIPTION OF THE DRAWINGS

These and other features of the invention will become more apparent in the following detailed description in which reference is made to the appended drawings wherein:

FIG. 1 presents a multiple sequence alignment showing conserved regions of a portion of the phoP gene from various Salmonella species. Shaded blocks highlight the following regions: bases 22 to 39: forward primer SEQ ID NO:32; bases 109 to 133: binding site for molecular beacon #2 [SEQ ID NO:34]; bases 142 to 159: reverse primer [SEQ ID NO:33];

FIG. 2 presents the arrangement of PCR primers and a molecular beacon probe on the phoP gene sequence in one embodiment of the invention. Numbers in parentheses indicate the positions of the first and last nucleotides of each feature on the PCR product generated with primers SEQ ID NOs:32 & 33;

FIG. 3 presents the secondary structure of a molecular beacon probe in accordance with one embodiment of the invention [SEQ ID NO:34]; and

FIG. 4 presents (A) the sequence of a Salmonella phoP gene [SEQ ID NO:1], and (B) the sequence of a conserved region (consensus sequence) of the Salmonella phoP gene, which is unique to Salmonella phoP gene isolates [SEQ ID NO:30].

### DETAILED DESCRIPTION OF THE INVENTION

The present invention is based on the identification of a highly conserved region (consensus sequence) that is common to various Salmonella species. The consensus sequence constitutes a suitable target sequence for the design of primers and probes capable of specifically amplifying and detecting Salmonella species in a test sample.

The present invention provides for primer and probe sequences capable of amplifying and/or detecting all or part of the consensus sequence that are suitable for use in detecting the presence of Salmonella bacteria in a range of samples including, but not limited to, clinical samples, microbiological pure cultures, food, and environmental and pharmaceutical quality control processes. In accordance with one embodiment of the present invention, the primers and probes are capable of amplifying and/or detecting target nucleic acid sequences from all seven known species of Salmonella, i.e. S. bongori, S. choleraesuis, S. enterica, S. enteritidis, S. paratyphi, S. typhi and S. typhimurium.

In another embodiment, the invention provides diagnostic assays that can be carried out in real time and addresses the need for rapid detection of Salmonella bacteria in a variety of biological samples.

### DEFINITIONS

Unless defined otherwise, all technical and scientific terms used herein have the same meaning as commonly understood by one of ordinary skill in the art to which this invention belongs.

The terms “oligonucleotide” and “polynucleotide” as used interchangeably herein refer to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The terms include polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

The terms “primer” and “polynucleotide primer,” as used herein, refer to a short, single-stranded polynucleotide capable of hybridizing to a complementary sequence in a nucleic acid sample. A primer serves as an initiation point for template-dependent nucleic acid synthesis. Nucleotides are added to a primer by a nucleic acid polymerase in accordance with the sequence of the template nucleic acid strand. A “primer pair” or “primer set” refers to a set of primers including a 5′ upstream primer that hybridizes with the 5′ end of the sequence to be amplified and a 3′ downstream primer that hybridizes with the complementary 3′ end of the sequence to be amplified. The term “forward primer” as used herein, refers to a primer which anneals to the 5′ end of the sequence to be amplified. The term “reverse primer”, as used herein, refers to a primer which anneals to the complementary 3′ end of the sequence to be amplified.

The terms “probe” and “polynucleotide probe,” as used herein, refer to a polynucleotide used for detecting the presence of a specific nucleotide sequence in a sample. Probes specifically hybridize to a target nucleotide sequence, or the complementary sequence thereof, and may be single- or double-stranded.

The term “specifically hybridize,” as used herein, refers to the ability of a polynucleotide to bind detectably and specifically to a target nucleotide sequence. Polynucleotides, oligonucleotides and fragments thereof specifically hybridize to target nucleotide sequences under hybridization and wash conditions that minimize appreciable amounts of detectable binding to non-specific nucleic acids. High stringency conditions can be used to achieve specific hybridization conditions as is known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

The term “corresponding to” refers to a polynucleotide sequence that is identical to all or a portion of a reference polynucleotide sequence. In contradistinction, the term “complementary to” is used herein to indicate that a polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The terms “hairpin” or “hairpin loop” refer to a single strand of DNA or RNA, the ends of which comprise complementary sequences, whereby the ends anneal together to form a “stem” and the region between the ends is not annealed and forms a “loop.” Some probes, such as molecular beacons, have such “hairpin” structure when not hybridized to a target sequence. The loop is a single-stranded structure containing sequences complementary to the target sequence, whereas the stem self-hybridises to form a double-stranded region. While the stem sequences are typically unrelated to the target sequence, nucleotides that are both complementary to the target sequence and that can self-hybridise can be included in the stem region, if desired.

The terms “target sequence” or “target nucleotide sequence,” as used herein, refer to a particular nucleic acid sequence in a test sample to which a primer and/or probe is intended to specifically hybridize. A “target sequence” is typically longer than the primer or probe sequence and thus can contain multiple “primer target sequences” and “probe target sequences.” A target sequence may be single or double stranded. The term “primer target sequence” as used herein refers to a nucleic acid sequence in a test sample to which a primer is intended to specifically hybridize. The term “probe target sequence” refers to a nucleic acid sequence in a test sample to which a probe is intended to specifically hybridize.

As used herein, the term “about” refers to a ±10% variation from the nominal value. It is to be understood that such a variation is always included in any given value provided herein, whether or not it is specifically referred to.

Target Sequence

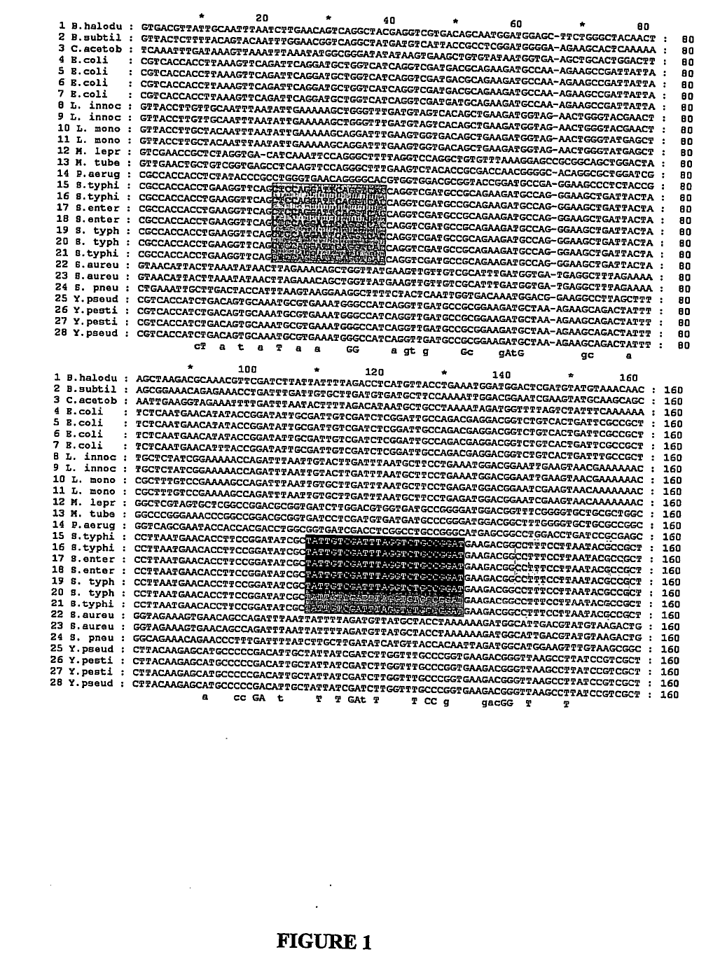

In order to identify regions of the Salmonella phoP gene that are highly conserved across Salmonella species and thus can potentially serve as target sequences for specific probes, phoP gene sequences (having a general sequence corresponding to SEQ ID NO:1) from a number of Salmonella species were subjected to a multiple sequence alignment analysis. A portion of a representative alignment is shown in FIG. 1. A 137 nucleotide region of the Salmonella phoP gene sequence, having a sequence corresponding to SEQ ID NO:30, was identified as being generally conserved in Salmonella species. This sequence is referred to herein as a consensus sequence.

Accordingly, the present invention provides an isolated Salmonella specific polynucleotide consisting of the consensus sequence as set forth in SEQ ID NO:30, or the complement thereof, that can be used as a target sequence for the design of probes for the specific detection of Salmonella.

It will be recognised by those skilled in the art that all, or a portion, of the consensus sequence set forth in SEQ ID NO:30 can be used as a target sequence for the specific detection of Salmonella. Thus, in one embodiment of the invention, a target sequence suitable for the specific detection of Salmonella comprising at least 60% of the sequence set forth in SEQ ID NO:30, or the complement thereof, is provided. In another embodiment, the target sequence comprises at least 65% of the sequence set forth in SEQ ID NO:30, or the complement thereof. In a further embodiment, the target sequence comprises at least 70% of the sequence set forth in SEQ ID NO:30, or the complement thereof. Target sequences comprising at least 75%, at least 85%, at least 90%, at least 95% and at least 98% of the sequence set forth in SEQ ID NO:30, or the complement thereof, are also contemplated.

Alternatively, such portions of the consensus sequence can also be expressed in terms of consecutive nucleotides of the sequence set forth in SEQ ID NO:30. Accordingly, target sequences comprising portions of the consensus sequence including at least 60, at least 65, at least 70, at least 75, at least 80, at least 85, at least 90, at least 95, at least 100, at least 105, at least 110 and at least 115 consecutive nucleotides of the sequence set forth in SEQ ID NO:30, or the complement thereof, are contemplated. By “at least 60 consecutive nucleotides” it is meant that the target sequence may comprise any number of consecutive nucleotides between 60 and 137 of the sequence set forth in SEQ ID NO:30, thus this range includes portions of the consensus sequence that comprise at least 61, at least 62, at least 63, at least 64, etc, consecutive nucleotides of the sequence set forth in SEQ ID NO:30.

Within the 137 nucleotide consensus sequence, two additional highly conserved regions were identified. These regions have sequences corresponding to SEQ ID NOs:31 and 39. Accordingly, one embodiment of the present invention provides for target sequences that comprise all or a portion of a sequence corresponding to SEQ ID NO:31 or 39, or the complement thereof.

It will also be appreciated that the target sequence may include additional nucleotide sequences that are found upstream and/or downstream of the consensus sequence in the Salmonella genome. As the assays provided by the present invention typically include an amplification step, it may be desirable to select an overall length for the target sequence such that the assay can be conducted fairly rapidly. Thus, the target sequence to be amplified typically has an overall length of less than about 500 nucleotides. In one embodiment, the target sequence has an overall length of less than about 400 nucleotides. In other embodiments, the target sequence has an overall length of less than about 350 nucleotides and less than about 300 nucleotides.

For assays that utilise molecular beacons, shorter target sequences may be appropriate, for example, less than about 250 nucleotides (see, for example, Mhlanga & Malmberg, (2001) Methods 25:463471). Thus, in one embodiment, the target sequence to be amplified for an assay utilising a molecular beacon is less than about 200 nucleotides in length. In another embodiment, the target sequence to be amplified is less than about 150 nucleotides in length. In a further embodiment, the target sequence to be amplified has an overall length of less than or equal to about 140 nucleotides.

Polynucleotide Primers and Probes

The present invention provides for polynucleotides for the amplification and/or detection of nucleic acids from one or more Salmonella species in a sample. The polynucleotides of the invention comprise a sequence that corresponds to or is complementary to a portion of the Salmonella phoP gene sequence and are capable of specifically hybridizing to Salmonella nucleic acids. In one embodiment, the polynucleotides of the invention comprise a sequence that corresponds to or is complementary to a portion of the Salmonella phoP gene sequence as set forth in SEQ ID NO:1. In a further embodiment, the polynucleotides of the invention comprise a sequence that corresponds to or is complementary to a portion of any one of the regions of the Salmonella phoP gene sequences as set forth in SEQ ID NOs:16 to 22 (shown in FIG. 1, numbered as 15-21, respectively).

The polynucleotides of the present invention are generally between about 7 and about 100 nucleotides in length. One skilled in the art will understand that the optimal length for a selected polynucleotide will vary depending on its intended application (i.e. primer, probe or combined primer/probe) and on whether any additional features, such as tags, self-complementary “stems” and labels (as described below), are to be incorporated. In one embodiment of the present invention, the polynucleotides are between about 10 and about 100 nucleotides in length. In another embodiment, the polynucleotides are between about 12 and about 100 nucleotides in length. In other embodiments, the polynucleotides are between about 12 and about 50 nucleotides and between 12 and 40 nucleotides in length.

One skilled in the art will also understand that the entire length of the polynucleotide primer or probe does not need to correspond to or be complementary to the Salmonella phoP gene sequence in order to specifically hybridize thereto. Thus, the polynucleotide primers and probes may comprise nucleotides at the 5′ and/or 3′ termini that are not complementary to the Salmonella phoP gene sequence. Such non-complementary nucleotides may provide additional functionality to the primer/probe, for example, they may provide a restriction enzyme recognition sequence or a “tag” that facilitates detection, isolation or purification. Alternatively, the additional nucleotides may provide a self-complementary sequence that allows the primer/probe to adopt a hairpin configuration. Such configurations are necessary for certain probes, for example, molecular beacon and Scorpion probes. Typically, the polynucleotide primers and probes of the invention comprise a sequence of at least 7 consecutive nucleotides that correspond to or are complementary to a portion of the Salmonella phoP gene sequence. As is known in the art, the optimal length of the sequence corresponding or complementary to the Salmonella phoP gene sequence will be dependent on the specific application for the polynucleotide, for example, whether it is to be used as a primer or a probe and, if the latter, the type of probe. Optimal lengths can be readily determined by the skilled artisan.

In one embodiment, the polynucleotides comprise at least 10 consecutive nucleotides corresponding or complementary to a portion of the Salmonella phoP gene sequence. In another embodiment, the polynucleotides comprise at least 12 consecutive nucleotides corresponding or complementary to a portion of the Salmonella phoP gene sequence. In a further embodiment, the polynucleotides comprise at least 15 consecutive nucleotides corresponding or complementary to a portion of the Salmonella phoP gene sequence. Polynucleotides comprising at least 18, at least 20, at least 22 and at least 24 consecutive nucleotides corresponding or complementary to a portion of the Salmonella phoP gene sequence are also contemplated.

Sequences of exemplary polynucleotides of the invention are set forth in Table 1. Further non-limiting examples for the polynucleotides of the invention include polynucleotides that comprise at least 7 consecutive nucleotides of any one of SEQ ID NOs: 30, 32, 33, 35, 37, 39 or 41.

TABLE 1
Exemplary polynucleotides of the invention

| | SEQ |
|---|---|
| | ID |
| Nucleotide sequence | NO |
| 5′-CTCCAGGATTCAGGTCAC-3′ | 32 |
| 5′-CGGCGTATTAAGGAAAGG-3′ | 33 |
| 5′-TATTGTCGATTTAGGTCTGCCGGAT-3′ | 35 |
| 5′-ATCCGGCAGACCTAAATCGACAATA-3′ | 37 |
| 5′-TGAACACCTTCCGGATATCGCTAT-3′ | 39 |
| 5′-ATAGCGATATCCGGAAGGTGTTCA-3′ | 41 |

Primers

As indicated above, the polynucleotide primers of the present invention comprise a sequence that corresponds to or is complementary to a portion of the Salmonella phoP gene sequence. In accordance with the invention, the primers are capable of amplifying a target nucleotide sequence comprising all or a portion of the 137 nucleotide consensus sequence as shown in SEQ ID NO:30. Accordingly, the present invention provides for primer pairs capable of amplifying a Salmonella target nucleotide sequence, wherein the target sequence is less than about 500 nucleotides in length and comprises at least 60 consecutive nucleotides of SEQ ID NO:30, or the complement thereof, as described above.

Thus, pairs of primers can be selected to comprise a forward primer corresponding to a portion of the Salmonella phoP gene sequence upstream of or within the region of the gene corresponding to SEQ ID NO:30 and a reverse primer that it is complementary to a portion of the Salmonella phoP gene sequence downstream of or within the region of the gene corresponding to SEQ ID NO:30. In accordance with the present invention, the primers comprise at least 7 consecutive nucleotides of the sequence set forth in SEQ ID NO:1, or the complement thereof. In one embodiment, the primers comprise at least 7 consecutive nucleotides of the sequence as set forth in any one of SEQ ID NOs:16-22, or the complement thereof. In another embodiment, the primers comprise at least 7 consecutive nucleotides of the sequence set forth in SEQ ID NO:30, or the complement thereof.

Appropriate primer pairs can be readily determined by a worker skilled in the art. In general, primers are selected that specifically hybridize to a portion of the Salmonella phoP gene sequence without exhibiting significant hybridization to non-Salmonella phoP nucleic acids. In addition, the primers are selected to contain minimal sequence repeats and such that they show the least likelihood of dimer formation, cross dimer formation, hairpin structure formation and cross priming. Such properties can be determined by methods known in the art, for example, using the computer modelling program OLIGO® Primer Analysis Software (distributed by National Biosciences, Inc., Plymouth, Minn.).

Non-limiting examples of suitable primer sequences include SEQ ID NOs: 32 and 33 shown in Table 1, as well as primers comprising at least 7 consecutive nucleotides of any one of SEQ ID NOs: 32, 33, 35, 37, 39 or 41.

Probes

In order to specifically detect one or more Salmonella species, the probe polynucleotides of the invention are designed to correspond to or be complementary to a portion of the Salmonella phoP gene consensus sequence shown in SEQ ID NO:30. The probe polynucleotides, therefore, comprise at least 7 consecutive nucleotides of the sequence set forth in SEQ ID NO:30, or the complement thereof. As indicated above, two highly conserved regions were identified within the Salmonella consensus sequence. In one embodiment, therefore, the present invention provides for probe polynucleotides comprising at least 7 consecutive nucleotides of the sequence set forth in SEQ ID NO:31 or 39, or the complement thereof.

Non-limiting examples of suitable probe sequences include SEQ ID NOs: 35, 37, 39 and 41 as shown in Table 1, as well as probes comprising at least 7 consecutive nucleotides of any one of SEQ ID NOs: 32, 33, 35, 39 or 41, or the complement thereof.

Various types of probes known in the art are contemplated by the present invention. For example, the probe may be a hybridization probe, the binding of which to a target nucleotide sequence can be detected using a general DNA binding dye such as ethidium bromide, SYBR® Green, SYBR® Gold and the like. Alternatively, the probe can incorporate one or more detectable labels. Detectable labels are molecules or moieties a property or characteristic of which can be detected directly or indirectly and are chosen such that the ability of the probe to hybridize with its target sequence is not affected. Methods of labelling nucleic acid sequences are well-known in the art (see, for example, Ausubel et al., (1997 & updates) Current Protocols in Molecular Biology, Wiley & Sons, New York).

Labels suitable for use with the probes of the present invention include those that can be directly detected, such as radioisotopes, fluorophores, chemiluminophores, enzymes, colloidal particles, fluorescent microparticles, and the like. One skilled in the art will understand that directly detectable labels may require additional components, such as substrates, triggering reagents, light, and the like to enable detection of the label. The present invention also contemplates the use of labels that are detected indirectly. Indirectly detectable labels are typically specific binding members used in conjunction with a “conjugate” that is attached or coupled to a directly detectable label. Coupling chemistries for synthesising such conjugates are well-known in the art and are designed such that the specific binding property of the specific binding member and the detectable property of the label remain intact. As used herein, “specific binding member” and “conjugate” refer to the two members of a binding pair, i.e. two different molecules, where the specific binding member binds specifically to the probe, and the “conjugate” specifically binds to the specific binding member. Binding between the two members of the pair is typically chemical or physical in nature. Examples of such binding pairs include, but are not limited to, antigens and antibodies; avidin/streptavidin and biotin; haptens and antibodies specific for haptens; complementary nucleotide sequences; enzyme cofactors/substrates and enzymes; and the like.

In one embodiment of the present invention, the probe is labelled with a fluorophore. The probe may additionally incorporate a quencher for the fluorophore. Fluorescently labelled probes can be particularly useful for the real-time detection of target nucleotide sequences in a test sample. Examples of probes that are labelled with both a fluorophore and a quencher that are contemplated by the present invention include, but are not limited to, molecular beacon probes and TaqMan® probes. Such probes are well known in the art (see for example, U.S. Pat. Nos. 6,150,097; 5,925,517 and 6,103,476; Marras et al., “Genotyping single nucleotide polymorphisms with molecular beacons.” In Kwok, P. Y. (ed.), “Single nucleotide polymorphisms: methods and protocols,” Vol. 212, pp. 111-128, Humana Press, Totowa, N.J.)

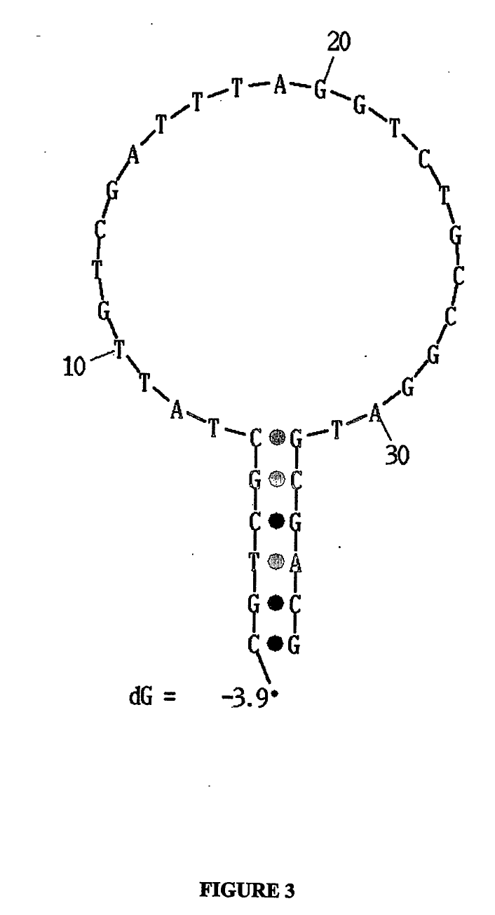

A molecular beacon probe is a hairpin shaped oligonucleotide sequence, which undergoes a conformational change when it hybridizes to a perfectly complementary target sequence. The secondary structure of a typical molecular beacon probe includes a loop sequence, which is capable of hybridizing to a target sequence and a pair of arm sequences. One “arm” of the probe sequence is attached to a fluorophore, while the other “arm” of the probe is attached to a quencher. The arm sequences are complementary to each other and hybridize together to form a molecular duplex such that the molecular beacon adopts a hairpin conformation. In this conformation, the fluorophore and quencher are in close proximity and interact such that emission of fluorescence is prevented. The loop sequence remains un-hybridized. Hybridization between the loop sequence and the target sequence forces the molecular beacon probe to undergo a conformational change in which arm sequences are forced apart and the fluorophore is physically separated from the quencher. As a result, the fluorescence of the fluorophore is restored. The fluorescence generated can be monitored and related to the presence of the target nucleotide sequence. If no target sequence is present in the sample, no fluorescence will be observed. This methodology, as described further below, can also be used to quantify the amount of target nucleotide in a sample. By way of example, FIG. 3 depicts the secondary structure of an exemplary hairpin loop molecular beacon (molecular beacon #2) having a sequence corresponding to SEQ ID NO:34 and a loop sequence corresponding to SEQ ID NO: 35.

Wavelength-shifting molecular beacon probes which incorporate two fluorophores, a “harvester fluorophore and an “emitter” fluorophore (see, Kramer, et al., (2000) Nature Biotechnology, 18:1191-1196) are also contemplated. When a wavelength-shifting molecular beacon binds to its target sequence and the hairpin opens, the energy absorbed by the harvester fluorophore is transferred by fluorescence resonance energy transfer (FRET) to the emitter, which then fluoresces. Wavelength-shifting molecular beacons are particularly suited to multiplex assays.

TaqMan® probes are dual-labelled fluorogenic nucleic acid probes that function on the same principles as molecular beacons. TaqMan® probes are composed of a polynucleotide that is complementary to a target sequence and is labelled at the 5′ terminus with a fluorophore and at the 3′ terminus with a quencher. TaqMan® probes, like molecular beacons, are typically used as real-time probes in amplification reactions. In the free probe, the close proximity of the fluorophore and the quencher ensures that the fluorophore is internally quenched. During the extension phase of the amplification reaction, the probe is cleaved by the 5′ nuclease activity of the polymerase and the fluorophore is released. The released fluorophore can then fluoresce and produce a detectable signal.

Linear probes comprising a fluorophore and a high efficiency dark quencher, such as the Black Hole Quenchers (BHQ™; Biosearch Technologies, Inc., Novato, Calif.) are also contemplated. As is known in the art, the high quenching efficiency and lack of native fluorescence of the BHQ™ dyes allows “random-coil” quenching to occur in linear probes labelled at one terminus with a fluorophore and at the other with a BHQ™ dye thus ensuring that the fluorophore does not fluoresce when the probe is in solution. Upon binding its target sequence, the probe stretches out spatially separating the fluorophore and quencher and allowing the fluorophore to fluoresce. One skilled in the art will appreciate that the BHQ™ dyes can also be used as the quencher moiety in molecular beacon or TaqMan® probes.

As an alternative to including a fluorophore and a quencher in a single molecule, two fluorescently labelled probes that anneal to adjacent regions of the target sequence can be used. One of these probes, a donor probe, is labelled at the 3′ end with a donor fluorophore, such as fluorescein, and the other probe, the acceptor probe, is labelled at the 5′ end with an acceptor fluorophore, such as LC Red 640 or LC Red 705. When the donor fluorophore is stimulated by the excitation source, energy is transferred to the acceptor fluorophore by FRET resulting in the emission of a fluorescent signal.

In addition to providing primers and probes as separate molecules, the present invention also contemplates polynucleotides that are capable of functioning as both primer and probe in an amplification reaction. Such combined primer/probe polynucleotides are known in the art and include, but are not limited to, Scorpion probes, duplex Scorpion probes, Lux™ primers and Amplifluor™ primers.

Scorpion probes consist of, from the 5′ to 3′ end, (i) a fluorophore, (ii) a specific probe sequence that is complementary to a portion of the target sequence and is held in a hairpin configuration by complementary stem loop sequences, (iii) a quencher, (iv) a PCR blocker (such as, hexethylene glycol) and (v) a primer sequence. After extension of the primer sequence in an amplification reaction, the probe folds back on itself so that the specific probe sequence can bind to its complement within the same DNA strand. This opens up the hairpin and the fluorophore can fluoresce. Duplex Scorpion probes are a modification of Scorpion probes in which the fluorophore-coupled probe/primer containing the PCR blocker and the quencher-coupled sequence are provided as separate complementary polynucleotides. When the two polynucleotides are hybridized as a duplex molecule, the fluorophore is quenched. Upon dissociation of the duplex when the primer/probe binds the target sequence, the fluorophore and quencher become spatially separated and the fluorophore fluoresces.

The Amplifluor Universal Detection System also employs fluorophore/quencher combinations and is commercially available from Chemicon International (Temecula, Calif.).

In contrast, Lux™ primers incorporate only a fluorophore and adopt a hairpin structure in solution that allows them to self-quench. Opening of the hairpin upon binding to a target sequence allows the fluorophore to fluoresce.

Suitable fluorophores and/or quenchers for use with the polynucleotides of the present invention are known in the art (see for example, Tgayi et al., Nature Biotechnol., 16:49-53 (1998); Marras et al., Genet. Anal.: Biomolec. Eng., 14:151-156 (1999)). Many fluorophores and quenchers are available commercially, for example from Molecular Probes (Eugene, Oreg.) or Biosearch Technologies, Inc. (Novato, Calif.). Examples of fluorophores that can be used in the present invention include, but are not limited to, fluorescein and fluorescein derivatives, such as 6-carboxyfluoroscein (FAM), 5′-tetrachlorofluorescein phosphoroamidite (ITET), tetrachloro-6-carboxyfluoroscein, VIC and JOE, 5-(2′-aminoethyl)aminonaphthalene-1-sulphonic acid (EDANS), coumarin and coumarin derivatives, Lucifer yellow, Texas red, tetramethylrhodamine, 5-carboxyrhodamine, cyanine dyes (such as Cy5) and the like. Pairs of fluorophores suitable for use as FRET pairs include, but are not limited to, fluorescein/rhodamine, fluorescein/Cy5, fluorescein/Cy5.5, fluorescein/LC Red 640, fluorescein/LC Red 750, and phycoerythrin/Cy7. Quenchers include, but are not limited to, 4′-(4-dimethylaminophenylazo)benzoic acid (DABCYL), 4-dimethylaminophenylazophenyl-4′-maleimide (DABMI), tetramethylrhodamine, carboxytetramethylrhodamine (TAMRA), BHQ™ dyes and the like.

Methods of selecting appropriate sequences for and preparing the various primers and probes are known in the art. For example, the polynucleotides can be prepared using conventional solid-phase synthesis using commercially available equipment, such as that available from Applied Biosystems USA Inc. (Foster City, Calif.), DuPont, (Wilmington, Del.), or Milligen (Bedford, Mass.). Methods of coupling fluorophores and quenchers to nucleic acids are also in the art.

In one embodiment of the present invention, the probe polynucleotide is a molecular beacon. In general, in order to form a hairpin structure effectively, molecular beacons are at least 17 nucleotides in length. In accordance with this aspect of the invention, therefore, the molecular beacon probe is typically between about 17 and about 40 nucleotides in length. Within the probe, the loop sequence that corresponds to or is complementary to the target sequence typically is about 7 to about 32 nucleotides in length, while the stem (or “arm”) sequences are each between about 4 and about 9 nucleotides in length. As indicated above, part of the stem sequences of a molecular beacon may also be complementary to the target sequence. In one embodiment of the present invention, the loop sequence of the molecular beacon is between about 10 and about 30 nucleotides in length. In other embodiments, the loop sequence of the molecular beacon is between about 15 and about 30 nucleotides in length.

In accordance with the present invention, the loop region of the molecular beacon probe comprises at least 7 consecutive nucleotides of the sequence as set forth in SEQ ID NO:30, or the complement thereof. In a specific embodiment, the loop region of the molecular beacon probe comprises at least 7 consecutive nucleotides of the sequence as set forth in SEQ ID NOs:31 or 39, or the complement thereof.

Amplification and Detection

In accordance with one embodiment of the present invention, Salmonella detection involves subjecting a test sample to an amplification reaction in order to obtain an amplification product, or “amplicon” comprising the target sequence.

As used herein, an “amplification reaction” refers to a process that increases the number of copies of a particular nucleic acid sequence by enzymatic means. Amplification procedures are well-known in the art and include, but are not limited to, polymerase chain reaction (PCR), TMA, rolling circle amplification, nucleic acid sequence based amplification (NASBA), strand displacement amplification (SDA) and Q-beta replicase amplification. One skilled in the art will understand that for use in certain amplification techniques the primers described above may need to be modified, for example, SDA primers comprise additional nucleotides near the 5′ end that constitute a recognition site for a restriction endonuclease. Similarly, NASBA primers comprise additional nucleotides near the 5′ end that are not complementary to the target sequence but which constitute an RNA polymerase promoter. Polynucleotides thus modified are considered to be within the scope of the present invention.

In one embodiment of the present invention, the target sequence is amplified by PCR. PCR is a method known in the art for amplifying a nucleotide sequence using a heat stable polymerase and a pair of primers, one primer (the forward primer) complementary to the (+)-strand at one end of the sequence to be amplified and the other primer (the reverse primer) complementary to the (−)-strand at the other end of the sequence to be amplified. Newly synthesized DNA strands can subsequently serve as templates for the same primer sequences and successive rounds of strand denaturation, primer annealing, and strand elongation, produce rapid and highly specific amplification of the target sequence. PCR can thus be used to detect the existence of a defined sequence in a DNA sample. The term “PCR” as used herein refers to the various forms of PCR known in the art including, but not limited to, quantitative PCR, reverse-transcriptase PCR, real-time PCR, hot start PCR, long PCR, LAPCR, multiplex PCR, touchdown PCR, and the like. “Real-time PCR” refers to a PCR reaction in which the amplification of a target sequence is monitored in real time by, for example, the detection of fluorescence emitted by the binding of a labelled probe to the amplified target sequence.

The present invention thus provides for amplification of a portion of a Salmonella phoP gene of less than about 500 nucleotides in length and comprising at least 60 consecutive nucleotides of the sequence set forth in SED ID NO:30 using pairs of polynucleotide primers, each member of the primer pair comprising at least 7 nucleotides of the sequence as set forth in SEQ ID NO:1, or the complement thereof.

The product of the amplification reaction can be detected by a number of means known to individuals skilled in the art. Examples of such detection means include, for example, gel electrophoresis and/or the use of polynucleotide probes. In one embodiment of the invention, the amplification products are detected through the use of polynucleotide probes. Such polynucleotide probes are described in detail above.

A further embodiment of the invention, therefore, provides for amplification and detection of a portion of a Salmonella phoP gene of less than about 500 nucleotides in length and comprising at least 60 consecutive nucleotides of the sequence set forth in SED ID NO:30 using a combination of polynucleotides, the combination comprising one or more polynucleotide primers comprising at least 7 nucleotides of the sequence as set forth in SEQ ID NO:1, or the complement thereof, and a polynucleotide probe comprising at least 7 consecutive nucleotides of the sequence as set forth in SEQ ID NO:30, or the complement thereof.

It will be readily appreciated that a procedure that allows both amplification and detection of target Salmonella nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real time” monitoring of the amplification reaction, as discussed above, as well as more conventional “end-point”, monitoring. In one embodiment, the detection is accomplished in real time in order to facilitate rapid detection. In a specific embodiment, detection is accomplished in real time through the use of a molecular beacon probe.

In one embodiment, the present invention thus provides for methods to specifically amplify and detect Salmonella nucleic acid sequences in a test sample in a single tube format using the polynucleotide primers, and optionally one or more probes, described herein. Such methods may employ dyes, such as SYBR® Green or SYBR® Gold that bind to the amplified target sequence, or an antibody that specifically detects the amplified target sequence. The dye or antibody is included in the reaction vessel and detects the amplified sequences as it is formed. Alternatively, a labelled polynucleotide probe (such as a molecular beacon or TaqMan® probe) distinct from the primer sequences, which is complementary to a region of the amplified sequence, may be included in the reaction, or one of the primers may act as a combined primer/probe, such as a Scorpion probe. Such options are discussed in detail above.

Thus, a general method of detecting Salmonella in a sample is provided that comprises contacting a test sample suspected of containing, or known to contain, a Salmonella target nucleotide sequence with a combination of polynucleotides comprising one or more polynucleotide primer and one or more polynucleotide probe or primer/probe, as described above, under conditions that permit amplification and detection of said target sequence, and detecting any amplified target sequence as an indication of the presence of Salmonella in the sample. A “test sample” as used herein is a biological sample suspected of containing, or known to contain, a Salmonella target nucleotide sequence.

In one embodiment of the present invention, a method using the polynucleotide primers and probes or primer/probes is provided to specifically amplify and detect a Salmonella target nucleotide sequence in a test sample, the method generally comprising the steps of:

(a) forming a reaction mixture comprising a test sample, amplification reagents, one or more labelled polynucleotide probe sequence capable of specifically hybridising to a portion of a Salmonella target nucleotide sequence and one or more polynucleotide primer corresponding to or complementary to a portion of a Salmonella phoP gene comprising said target nucleotide sequence;

(b) subjecting the mixture to amplification conditions to generate at least one copy of the target nucleotide sequence, or a nucleic acid sequence complementary to the target nucleotide sequence;

(c) hybridizing the probe to the target nucleotide sequence or the nucleic acid sequence complementary to the target sequence, so as to form a probe:target hybrid; and

(d) detecting the probe:target hybrid as an indication of the presence of the Salmonella target nucleotide sequence in the test sample.

The term “amplification reagents” includes conventional reagents employed in amplification reactions and includes, but is not limited to, one or more enzymes having nucleic acid polymerase activity, enzyme cofactors (such as magnesium or nicotinamide adenine dinucleotide (NAD)), salts, buffers, nucleotides such as deoxynucleotide triphosphates (dNTPs; for example, deoxyadenosine triphosphate, deoxyguanosine triphosphate, deoxycytidine triphosphate and deoxythymidine triphosphate) and other reagents that modulate the activity of the polymerase enzyme or the specificity of the primers.

It will be readily understood by one skilled in the art that step (b) of the above method can be repeated several times prior to step (c) by thermal cycling the reaction mixture by techniques known in the art and that steps (b), (c) and (d) may take place concurrently such that the detection of the amplified sequence takes place in real time. In addition, variations of the above method can be made depending on the intended application of the method, for example, the polynucleotide probe may be a combined primer/probe, or it may be a separate polynucleotide probe, in which case two different polynucleotide primers are used. Additional steps may be incorporated before, between or after those listed above as necessary, for example, the test sample may undergo enrichment, extraction and/or purification steps to isolate nucleic acids therefrom prior to the amplification reaction, and/or the amplified product may be submitted to purification/isolation steps or further amplification prior to detection, and/or the results from the detection step (d) may be analysed in order to quantify the amount of target present in the sample or to compare the results with those from other samples. These and other variations will be apparent to one skilled in the art and are considered to be within the scope of the present invention.

In one embodiment of the present invention, the method is a real-time PCR assay utilising two polynucleotide primers and a molecular beacon probe.

Diagnostic Assays to Detect Salmonella Species

The present invention provides for diagnostic assays using the polynucleotide primers and/or probes that can be used for highly specific detection of Salmonella in a test sample. The diagnostic assays comprise amplification and detection of Salmonella nucleic acids as described above. The diagnostic assays can be qualitative or quantitative and can involve real time monitoring of the amplification reaction or more conventional end-point monitoring.

In one embodiment, the invention provides for diagnostic assays that do not require post-amplification manipulations and minimise the amount of time required to conduct the assay. For example, in a specific embodiment, there is provided a diagnostic assay, utilising the primers and probes described herein, that can be completed using real time PCR technology in, at most, 54 hours and generally less that 24 hours.

Such diagnostic assays are particularly useful in the detection of Salmonella contamination of various foodstuffs. Thus, in one embodiment, the present invention provides a rapid and sensitive diagnostic assay for the detection of Salmonella contamination of a food sample. Foods that can be analysed using the diagnostic assays include, but are not limited to, dairy products such as milk, including raw milk, cheese, yoghurt, ice cream and cream; raw, cooked and cured meats and meat products, such as beef, pork, lamb, mutton, poultry (including turkey, chicken), game (including rabbit, grouse, pheasant, duck), minced and ground meat (including ground beef, ground turkey, ground chicken, ground pork); eggs; fruits and vegetables; nuts and nut products, such as nut butters; seafood products including fish and shellfish; and fruit or vegetable juices. The diagnostic assays may also be used to detect Salmonella contamination of drinking water.

While the primary focus of Salmonella detection is food products, the present invention also contemplates the use of the primers and probes in diagnostic assays for the detection of Salmonella contamination of other biological samples, such as patient specimens in a clinical setting, for example, faeces, blood, saliva, throat swabs, urine, mucous, and the like. The diagnostic assays are also useful in the assessment of microbiologically pure cultures, and in environmental and pharmaceutical quality control processes.

The test sample can be used in the assay either directly (i.e. as obtained from the source) or following one or more pre-treatment steps to modify the character of the sample. Thus, the test sample can be pre-treated prior to use, for example, by disrupting cells or tissue, enhancing/enriching the microbial content of the sample by culturing in a suitable medium, preparing liquids from solid materials, diluting viscous fluids, filtering liquids, distilling liquids, concentrating liquids, inactivating interfering components, adding reagents, purifying nucleic acids, and the like. In one embodiment of the present invention, the test sample is subjected to one or more steps to isolate, or partially isolate, nucleic acids therefrom.

As indicated above, the polynucleotide primers and probes of the invention can be used in assays to quantitate the amount of a Salmonella target nucleotide sequence in a test sample. Thus, the present invention provides for methods to specifically amplify, detect and quantitate a target nucleotide sequence in a test sample, the methods generally comprising the steps of:

(a) forming a reaction mixture comprising a test sample, amplification reagents, one or more labelled polynucleotide probe sequence capable of specifically hybridising to a portion of a Salmonella target nucleotide sequence and one or more polynucleotide primer corresponding to or complementary to a portion of an Salmonella phoP gene comprising said target nucleotide sequence;

(b) subjecting the mixture to amplification conditions to generate at least one copy of the target nucleotide sequence, or a nucleic acid sequence complementary to the target nucleotide sequence;

(c) hybridizing the probe to the target nucleotide sequence or the nucleic acid sequence complementary to the target sequence, so as to form a probe:target hybrid;

(d) detecting the probe:target hybrid by detecting the signal produced by the hybridized labelled probe; and

(e) analysing the amount of signal produced as an indication of the amount of target nucleotide sequence present in the test sample.

Step (e) can be conducted, for example, by comparing the amount of signal produced to a standard or utilising one of a number of statistical methods known in the art that do not require a standard.

The steps of this method may also be varied as described above for the amplification/detection method.

Various types of standards for quantitative assays are known in the art. For example, the standard can consist of a standard curve compiled by amplification and detection of known quantities of the Salmonella target nucleotide sequence under the assay conditions. Alternatively, relative quantitation can be performed without the need for a standard curve (see, for example, Pfaffl, M W. (2001) Nucleic Acids Research 29(9):2002-2007). In this method, a reference gene is selected against which the detection of the target gene can be compared. The reference gene is usually a gene that is expressed constitutively, for example, a house-keeping gene. An additional pair of primers and an appropriate probe are included in the reaction in order to amplify and detect a portion of the selected reference gene.

Another similar method of quantification is based on the inclusion of an internal standard in the reaction. Such internal standards generally comprise a control target nucleotide sequence and a control polynucleotide probe. The internal standard can further include an additional pair of primers that specifically amplify the control target nucleotide sequence and are unrelated to the polynucleotides of the present invention. Alternatively, the control target sequence can contain primer target sequences that allow specific binding of the assay primers but a different probe target sequence. This allows both the Salmonella target sequence and the control sequence to be amplified with the same primers, but the amplicons are detected with separate probe polynucleotides. Typically, when a reference gene or an internal standard is employed, the reference/control probe incorporates a detectable label that is distinct from the label incorporated into the Salmonella target sequence specific probe. The signals generated by these two labels when they bind their respective target sequences can thus be distinguished.

In the context of the present invention, a control target nucleotide sequence is a nucleic acid sequence that (i) can be amplified either by the Salmonella target sequence specific primers or by control primers, (ii) specifically hybridizes to the control probe under the assay conditions and (iii) does not exhibit significant hybridization to the Salmonella target sequence specific probe under the same conditions. One skilled in the art will recognise that the actual nucleic acid sequences of the control target nucleotide and the control probe are not important provided that they both meet the criteria outlined above.

The diagnostic assays can be readily adapted for high-throughput. High-throughput assays provide the advantage of processing many samples simultaneously and significantly decrease the time required to screen a large number of samples. The present invention, therefore, contemplates the use of the polynucleotides of the present invention in high-throughput screening or assays to detect and/or quantitate Salmonella target nucleotide sequences in a plurality of test samples.

For high-throughput assays, reaction components are usually housed in a multi-container carrier or platform, such as a multi-well microtitre plate, which allows a plurality of assays each containing a different test sample to be monitored simultaneously. Control samples can also be included in the plates to provide internal controls for each plate. Many automated systems are now available commercially for high-throughput assays, as are automation capabilities for procedures such as sample and reagent pipetting, liquid dispensing, timed incubations, formatting samples into microarrays, microplate thermocycling and microplate readings in an appropriate detector, resulting in much faster throughput times.

Kits and Packages for the Detection of Salmonella Species

The present invention further provides for kits for detecting Salmonella in a variety of samples. In general, the kits comprise a pair of primers and a probe capable of amplifying and detecting a Salmonella target sequence as described above. One of the primers and the probe may be provided in the form of a single polynucleotide, such as a Scorpion probe, as described above. The probe provided in the kit can incorporate a detectable label, such as a fluorophore or a fluorophore and a quencher, or the kit may include reagents for labelling the probe. The primers/probes can be provided in separate containers or in an array format, for example, pre-dispensed into microtitre plates.

The kits can optionally include amplification reagents, such as buffers, salts, enzymes, enzyme co-factors, nucleotides and the like. Other components, such as buffers and solutions for the enrichment, isolation and/or lysis of bacteria in a test sample, extraction of nucleic acids, purification of nucleic acids and the like may also be included in the kit. One or more of the components of the kit may be lyophilised and the kit may further comprise reagents suitable for the reconstitution of the lyophilised components.

The various components of the kit are provided in suitable containers. As indicated above, one or more of the containers may be a microtitre plate. Where appropriate, the kit may also optionally contain reaction vessels, mixing vessels and other components that facilitate the preparation of reagents or nucleic acids from the test sample.

The kit may additionally include one or more controls. For example, control polynucleotides (primers, probes, target sequences or a combination thereof) may be provided that allow for quality control of the amplification reaction and/or sample preparation, or that allow for the quantitation of Salmonella target nucleotide sequences.

The kit can additionally contain instructions for use, which may be provided in paper form or in computer-readable form, such as a disc, CD, DVD or the like.

The present invention further contemplates that the kits described above may be provided as part of a package that includes computer software to analyse data generated from the use of the kit.

The invention will now be described with reference to specific examples. It will be understood that the following examples are intended to describe preferred embodiments of the invention and are not intended to limit the invention in any way.

### EXAMPLES

### Example 1

### Determination of Unique, Conserved DNA Regions in Salmonella Species

The phoP gene coding regions from Salmonella species were sequenced and aligned using the multiple alignment program Clustal W™. The resulting alignment was used to identify short DNA regions that were conserved within the Salmonella genus, but which are excluded from other bacteria. FIG. 1 depicts a sample of such an alignment in which a portion of the phoP gene of 7 different Salmonella isolates has been aligned.

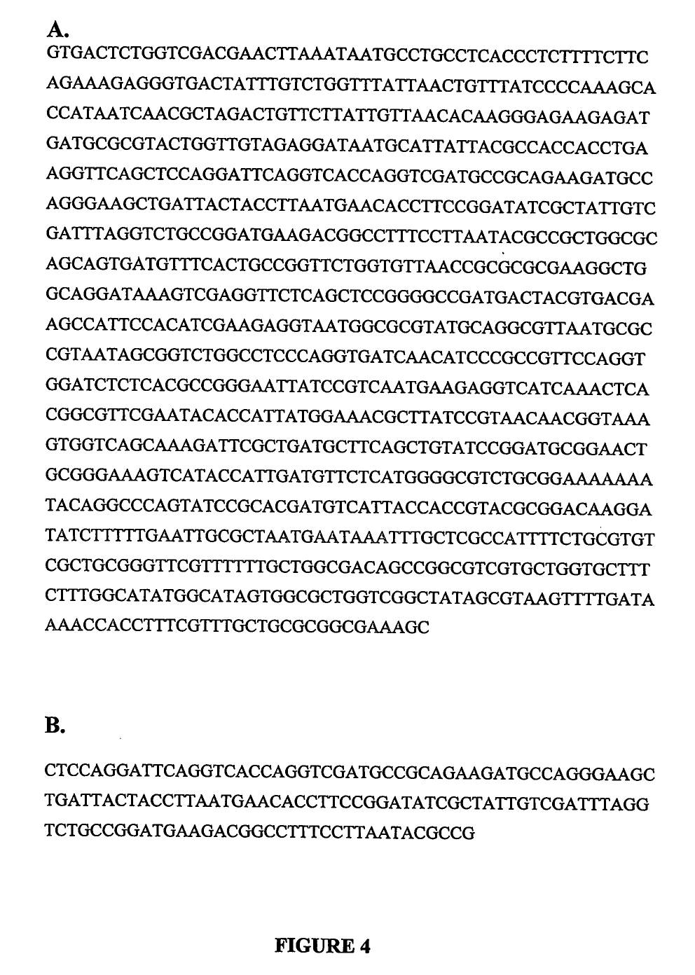

From the sequence of a Salmonella phoP gene (as shown in FIG. 4A; SEQ ID NO:1), a 137 nucleotide conserved sequence (consensus sequence) was identified as described above (shown in FIG. 4B, SEQ ID NO:30). This unique and conserved element of Salmonella phoP gene sequences was used to design highly specific primers for the PCR amplification of a conserved region of the Salmonella phoP gene.

### Example 2

### Generation of PCR Primers for Amplification of the Salmonella phoP Consensus Sequence

Within the conserved 137 nucleotide sequence identified as described in Example 1 two regions that could serve as primer target sequences were identified. These primer target sequences were used to design a pair of primers to allow efficient PCR amplification. The primer sequences are shown below:

Forward primer:

[SEQ ID NO:32]

5′-CTCCAGGATTCAGGTCAC-3′
Reverse primer:

[SEQ ID NO:33]

5′-CGGCGTATTAAGGAAAGG-3′

In the alignment presented in FIG. 1, the positions of the forward and reverse primers are represented by shaded boxes. The forward primer starts at position 22 and ends at position 39 of the alignment. The reverse primer represents the reverse complement of the region starting at position 142 and ending at position 159.

### Example 3

### Generation of Molecular Beacon Probes Specific for Salmonella Species

In order to design molecular beacon probes specific for Salmonella species, two regions within the phoP consensus sequence described above were identified which are not only was highly conserved in all Salmonella species but are also exclusive to Salmonella species. These sequences, which are suitable for use as a molecular beacon target sequences, are provided below:

[SEQ ID NO:31]

5′-TATTGTCGATTTAGGTCTGCCGGAT-3′

[SEQ ID NO:39]

5′-TGAACACCTTCCGGATATCGCTAT-3′

The complement of the above sequences are also suitable for use as a molecular beacon target sequences (SEQ ID NOs:37 and 41, respectively, shown below).

[SEQ ID NO:37]

5′-ATCCGGCAGACCTAAATCGACAATA-3′

[SEQ ID NO:41]

5′-ATAGCGATATCCGGAAGGTGTTCA-3′

Molecular beacon probes having the sequences shown below were synthesized by Integrated DNA Technologies Inc. Lowercase letter indicate stem sequences.

Molecular beacon probe #2:

[SEQ ID NO:34]

5′-cgtcgcTATTGTCGATTTAGGTCTGCCGGATgcgacg-3′
Molecular beacon probe #1:

[SEQ ID NO:38]

5′-cgacgcTGAACACCTTCCGGATATCGCTATgcgtcg-3′

The complement of the above sequences (SEQ ID NOs:36 and 40, respectively, shown below) can also be used as molecular beacon probes for detecting Salmonella.

[SEQ ID NO:36]

5′-cgtcgcATCCGGCAGACCTAAATCGACAATAgcgacg-3′

[SEQ ID NO:40]

5′-cgacgcATAGCGATATCCGGAAGGTGTTCAgcgtcg-3′

The starting material for the synthesis of the molecular beacons was an oligonucleotide that contains a sultfhydryl group at its 5′ end and a primary amino group at its 3′ end. DABCYL was coupled to the primary amino group utilizing an amine-reactive derivative of DABCYL. The oligonucleotides that were coupled to DABCYL were then purified. The protective trityl moiety was then removed from the 5′-sulfhydryl group and a fluorophore was introduced in its place using an iodoacetamide derivative.

An individual skilled in the art would recognize that a variety of methodologies could be used for synthesis of the molecular beacons. For example, a controlled-pore glass column that introduces a DABCYL moiety at the 3′ end of an oligonucleotide has recently become available, which enables the synthesis of a molecular beacon completely on a DNA synthesizer.

Table 2 provides a general overview of the characteristics of molecular beacon probe #2. The beacon sequence shown in Table 2 indicates the stem region in lower case and the loop region in upper case.

TABLE 2
Description of molecular beacon probe #2.

| | |
|---|---|
| Beacon sequence: | cgtcgcTATTGTCGATTTAGGTCTGCCGGATg |
| (5′→3′) | cgacg |
| | [SEQ ID NO:34] |
| Fluorophore (5′): | FAM |
| Quencher (3′): | DABCYL |

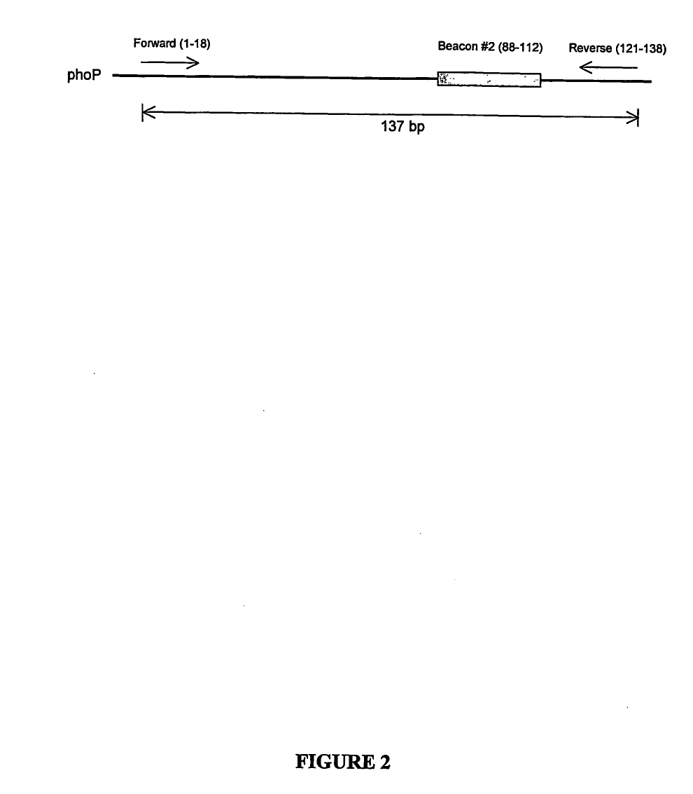

Table 3 provides an overview of the thermodynamics of the folding of molecular beacon probe #2. Calculations were made using MFOLD™ software, or the Oligo Analyzer software package available on Integrated DNA Technologies Inc. web site. FIG. 2 shows the arrangement of PCR primers and the molecular beacon probe in the Salmonella phoP consensus sequence. Numbers in parentheses indicate the positions of the first and last nucleotides of each feature on the PCR product generated with the forward and reverse primers.

TABLE 3A
Thermodynamics of molecular beacon probe #2.

| | | |
|---|---|---|
| Tm loop (thermodynamics algorithm) | 65.8° | C. |
| Tm stem (mFOLD calculation) | 61.7° | C. |
| ΔG₃₇ (mFOLD calculation) | −3.87 | kCal/mol |
| ΔH (mFOLD calculation) | −52.9 | kCal/mol |

TABLE 3B
Thermodynamics of molecular beacon probe #1.

| | | |
|---|---|---|
| Tm loop (thermodynamics algorithm) | 64.9° | C. |
| Tm stem (mFOLD calculation) | 62.4° | C. |
| ΔG₃₇ (mFOLD calculation) | −3.97 | kCal/mol |
| ΔH (mFOLD calculation) | −52.9 | kCal/mol |

### Example 4

### Isolation of DNA from Test Samples

The following protocol was utilized in order to isolate DNA sequences from samples. Material needed for DNA extraction:
Tungsten carbide beads: Qiagen
Reagent DX: Qiagen
DNeasy Plant Mini Kit: Qiagen
Tissue Disruption equipment: Mixer Mill™ 300 (Qiagen)

The following method was followed:
1) Add to a 2 ml screw top tube: 1 tungsten carbide bead and 0.1 g glass beads 212 to 300 μm in width+sample to be analysed+500 μL of API buffer+1 μL of Reagent DX+1 μL of RNase A (100 mg/mL). Extraction control done without adding sample to be analysed.
2) heat in Dry-Bath at 80° C. for 10 min.
3) mix in a Mixer Mill 300 (MM300) at frequency of 30 Hz [1/s], 2 min.
4) rotate tubes and let stand for 10 min at room temperature.
5) mix in a Mixer Mill 300, frequency 30 Hz, 2 min.
6) place tubes in boiling water for 5 min.
7) centrifuge with a quick spin.
8) add 150 μL of AP2 buffer.
9) mix at frequency of 30 Hz for 30 sec. Rotate tubes and repeat.
10) centrifuge at 13,000 rpm for 1 min.
11) place tubes at −20° C. for 10 min.
12) centrifuge at 13,000 rpm for 1 min.
13) transfer supernatant in to a 2 mL screw top tube containing 800 μL of AP3/E buffer.
14) mix by inverting, centrifuge with a quick spin.
15) add 700 μL of mixture. From step 13 to a DNeasy binding column and centrifuge at 800 rpm for 1 minute. Discard eluted buffer. Repeat process with leftover mixture from step 11.
16) add 500 μL of wash buffer (AW buffer) to binding columns and centrifuge for 1 minute at 800 rpm. Discard eluted buffer.
17) add 500 μL of wash buffer (AW buffer) to binding columns and centrifuge for 1 minute at 800 rpm. Discard eluted buffer.
18) centrifuge column again at 8000 rpm for 1 min.
19) place column in a sterile 2 mL tube and add 100 μL of AE elution buffer preheated at 80° C.
20) incubate for 1 min. Centrifuge at max speed for 2 min. Elute twice with 50 μL.
21) keep elution for PCR amplification.

Time of manipulation: 3 hours. Proceed to prepare PCR reaction for real-time detection.

### Example 5

### Amplification of a Target Sequence and Hybridization of Molecular Beacon Probe #2 in Real Time

PCR amplification was undertaken using the conditions described in Tables 4 and 5 below. The intensity of fluorescence emitted by the fluorophore component of the molecular beacon was detected at the annealing stage of each amplification cycle. In Table 4, note that the PCR buffer contains 1.5 mM magnesium chloride (final concentration). Inclusion of additional magnesium chloride brings the final concentration to 4 mM in the reaction mixture.

TABLE 4
PCR mix used for validation.

| | Final concentration in | |
|---|---|---|
| Reagent | reconstituted reaction | |
| Qiagen PCR buffer, 10× | 1× | |
| Forward primer [SEQ ID NO: 32], 2 μM | 0.4 | μM |
| Reverse primer [SEQ ID NO: 33], 2 μM | 0.4 | μM |
| dNTPs, 10 mM | 0.2 | mM |
| MgCl₂, 25 mM | 2.5 | mM |
| Molecular beacon [SEQ ID NO: 34], | 0.3 | μM |
| 10 μM | | |

| | |
|---|---|
| HotStartTaq, 5 U/μL | 1 U/25 μL reaction |

Table 5 presents an overview of the cycles used for each step of the PCR amplification.

TABLE 5
PCR program used throughout diagnostic test validation.

| Step | Temperature | Duration | | Repeats |
|---|---|---|---|---|
| Initial polymerase activation | 95° C. | 15 | min | 1 |
| Denaturation | 94° C. | 15 | sec | 40 |
| Annealing | 55° C. | 30 | sec | |
| Elongation | 72° C. | 30 | sec | |

Fluorescence was detected in real-time using a fluorescence monitoring real-time PCR instrument, for example, a BioRad iCycler i™ or MJ Research Opticon™. Other instruments with similar fluorescent reading abilities can also be used.

### Example 6

### Ouantification of Target Sequence in a Test Sample

In order to quantify the amount of target sequence in a sample, DNA was isolated and amplified as described in the preceding Examples (4 and 5). DNA was quantified using a standard curve constructed from serial dilutions of a target DNA solution of known concentration.

### Example 7

### Positive Validation for the Specificity of Molecular Beacon Probe #2 for Detection of Salmonella Species

The effectiveness of molecular beacon probe #2 for detecting Salmonella species was demonstrated as described generally below.

Genomic DNA from the species and strains presented in Table 6 below was isolated and amplified as described in the preceding Examples (4 and 5). Results are presented in Table 6 and indicate that molecular beacon probe #2 was capable of detecting all Salmonella species and strains tested.

In Table 6, figures in parentheses indicate the number of strains of each Salmonella species that were tested (if more than one). All strains gave a positive signal.

Similar results were obtained using forward and reverse primers with molecular beacon #1 under the conditions described in Example 5, except that this beacon gave one false negative signal under the conditions used in this assay (Salmonella bongori).

TABLE 6
Positive validation of molecular beacon probe
#2 and forward and reverse primers.

| | | | |
|---|---|---|---|
| Salmonella | Salmonella | Salmonella | Salmonella |
| enterica, subsp. | enteritidis (10) | paratyphi | enterica subsp. |
| enterica serovar | | (13) | enterica serovar |
| Agona | | | Thompson |
| Salmonella | Salmonella | Salmonella | Salmonella typhi |
| choleraesuis | enterica, subsp. | paratyphi | |
| subsp. arizonae | enterica serovar | type A | |
| (2) | Heidelberg | | |
| Salmonella | Salmonella | Salmonella | Salmonella |
| bongori (1) | enterica, subsp. | paratyphi | typhimurium (7) |
| | houtenae | type B | |
| Salmonella | Salmonella | Salmonella | Salmonella |
| enterica, subsp. | enterica subsp. | paratyphi | enterica subsp. |
| enterica serovar | indica | type C | enterica serovar |
| Brandenburg | | | Typhisuis |
| Salmonella | Salmonella | Salmonella | Salmonella spp |
| choleraesuis (5) | enterica subsp. | enterica | |
| | enterica serovar | subsp. | |
| | Infantis | enterica | |
| | | serovar | |
| | | Saintpaul | |
| Salmonella | Salmonella | Salmonella | |
| enterica, subsp. | enterica subsp. | enterica | |
| diarizonae | enterica serovar | subsp. | |
| | Montevideo | enterica | |
| | | serovar | |
| | | Senftenberg | |
| Salmonella | Salmonella | Salmonella | |
| enterica subsp. | enterica subsp. | enterica | |
| enterica serovar | enterica serovar | subsp. | |
| Dublin | Newport (3) | enterica | |
| | | serovar | |
| | | Stanley | |

### Example 8

### Negative Validation of the Primers and Molecular Beacon Probes

In order to test the ability of the molecular beacon probes to preferentially detect only Salmonella species, a number of bacteria from groups other than Salmonella were tested, as generally described below.

Samples of genomic DNA from the bacteria presented in Table 7 below were isolated as described in Example 4. PCR reactions were conducted using conditions and parameters as described in Example 5 but without the inclusion of the molecular beacon. SYBR® Green was used to detect the presence of any amplified products. No amplification products were observed for any of the species tested.

Additional rounds of tests were conducted including either molecular beacon probe #1 or #2. No hybridization of molecular beacon #2 or #1 was observed with any of the species tested.

In Table 7, the figures in parentheses indicate the number of strains of each species that were tested (if more than one). None of the tested strains provided a positive result with molecular beacon #2 or #1.

The above results suggest that both the amplification primers, and the molecular beacons are highly specific for Salmonella species.

TABLE 7
Negative Validation of the Primers and Molecular Beacon probes

| | | | |
|---|---|---|---|
| Acinetobacter | Chromobacterium | Kurthia zopfii | Pseudomonas |
| calcoaceticus | violaceum | | aeruginosa |
| Acinetobacter | Chryseomonas | Lactobacillus | Pseudomonas |
| junii | indologenes | acidophilus | alcaligenes |
| Aeromonas | Chryseomonas | Lactobacillus | Pseudomonas |
| hydrophila | luteola | casei | fragi |
| Aeromonas | Citrobacter | Lactobacillus | Pseudomonas |
| salmonicida | amalonaticus | delbreuckii | putida |
| Alcaligenes | Citrobacter | Lactobacillus | Pseudomonas |
| faecalis (2) | diversus | plantarum | stutzeri |
| Bacillus | Citrobacter | Lactococcus | Ralstonia |
| amylolique- | werkmanii | lactis | picketti |
| faciens (2) | | | |
| Bacillus | Clostridium | Legionella | Serratia |
| brevis | butyricum | micdadei | marcescens |
| Bacillus | Clostridium | Legionella | Shigella |
| cereus | difficile | pneumophila | dysenteriae |
| | | | (10) |
| Bacillus | Clostridium | Listeria | Shigella |
| circulans | perfringens | grayi | flexneri |
| Bacillus | Clostridium | Listeria | Shigella |
| firmus | sporogenes | innocua | sonnei |
| Bacillus | Clostridium | Listeria | Staphylococcus |
| lentus | tetani | ivanovii | aureus |
| Bacillus | Clostridium | Listeria | Staphylococcus |
| licheniformis | tyrobutyricum | monocytogenes | capitis |
| Bacillus | Corynebacterium | Listeria | Staphylococcus |
| megaterium | xerosis | seeligeri | epidermidis |
| Bacillus | Edwardsiella | Listeria | Staphylococcus |
| pumilus (5) | tarda | welshimeri | lentis |
| Bacillus | Enterobacter | Micrococcus | Stenotrophomonas |
| stearo- | aerogenes | luteus | maltophilia |
| thermophilus | | | |
| Bacillus | Enterobacter | Mycobacterium | Streptococcus |
| subtilis (2) | cloacae | smegmatis | agalactiae |
| Bacillus | Enterococcus | Neisseria | Streptococcus |
| thuringiensis | faecalis | gonorrhoeae | bovis |
| Bacteroides | Enterococcus | Neisseria | Streptococcus |
| fragilis | faecium | lactamica | mitis |
| Bordetella | Enterococcus | Neisseria | Streptococcus |
| bronchispetica | hirae | meningitidis | pneumoniae (2) |
| Bordetella | Erwinia | Neisseria | Streptococcus |
| pertussis | herbicola | sica | pyogenes |
| Borrelia | Escherichia | Nocardia | Streptococcus |
| burgdorferi | coli (3) | asteroides | suis |
| Branhamella | Haemophilus | Pediococcus | Yersinia |
| catarrhalis | influenzae | acidilactici | enterocolitica |
| Brevibacillus | Hafnia alvei | Proteus | |
| laterosporus | | mirabilis | |
| Campylobacter | Klebsiella | Proteus | |
| jejuni | pneumoniae | vulgaris | |
| Campylobacter | Kocuria | Pseudomonas | |
| rectus | kristinae | acidovorans | |

### Example 9

### Enrichment Procedure

A test sample can be submitted to non-selective enrichment steps (pre-enrichment) and/or selective enrichment prior to DNA extraction in order to enrich the bacterial content of the sample. The following is a representative protocol that can be followed (see, for example, Health Canada protocol MFHPB-20).

The following protocol can be followed for the pre-enrichment of the samples:
1) place 25 g or 25 mL of the sample in a stomacher bag, containing 225 mL of a suitable non-selective enrichment broth pH 6.0-7.0 (e.g. Nutrient broth, buffered peptone water or tryptone soy broth).
3) homogenize the bag contents with a Stomacher instrument.
4) incubate the stomacher bag at 35° C.±0.5° C. for 18-24 hr.
5) ensure that the contents in the stomacher bag are mixed properly to obtain a homogenous sample.
6) remove 10 μL or 1.0 ml of the enrichment broth and proceed to DNA extraction.

Proceed to isolate DNA from samples, for example using the procedure outlined in Example 4 or 10.

### Example 10

### Alternative DNA Extraction Protocol

Reagents required: Tungsten carbide beads: Qiagen
Reagent DX: Qiagen
DNeasy Mini Kit: Qiagen (including the following: lysis buffer (API), neutralization buffer (AP2), equilibration buffer (AP3/E), wash buffer (AW), elution buffer (AE) and RNase (100 mg/ml).
Tissue Disruption equipment: Mixer Mill™ 300 (Qiagen)

Protocol:
1) After enrichment as described in Example 9, 1 ml of resuspended cells are placed in a 2ml screw-cap centrifuge tube with a conical base.
2) Tubes are centrifuged at 6,000×g for 5 min. Supernatant is discarded. Some fat and food debris may remain. At this point, the cell pellet may be stored at −20° C. for up to 1 month before proceeding with the analysis.
3) Cell pellet is resuspended by vortexing with 500 μl lysis buffer and tungsten bead(s), then heated at 105° C. in a dry bath for 10 min. and allowed to cool to room temperature.
4) Tubes are placed in a Mixer Mill rack and shaken for 1 min. at 30 oscillations per sec. Tubes are rotated and the shaking step repeated.
5) A brief centrifugation (6,000×g for approx. 1 min.) is followed by addition of 200 μl neutralization buffer. Tubes are shaken in Mixer Mill rack for approx. 15 sec at 30 oscillations per sec. Tubes are rotated and the shaking step repeated. Tubes are centrifuged at 6,000×g for 5 min.
6) Supernatant is removed to a new tube containing 700 μl equilibration buffer and contents of tube are mixed by inverting then collected at bottom of tube by a brief centrifugation (6,000×g for approx. 1 min.).
7) 700 μl of the solution is transferred to a DNA binding column and centrifuged at 6,000×g for 1 min. Eluate is discarded. Centrifugation is repeated and any additional eluate discarded.

700 μl wash buffer is added to column and the column is centrifuged at 6,000×g for 1 min. Eluate is discarded. Centrifugation is repeated and any additional eluate discarded.
9) 400 μl elution buffer is added to column and allowed to stand for 1 min. Column is then centrifuged at 6,000×g for 1 min.
10) Eluate is retained for PCR analysis. 10 μl of eluate is suitable for use in the PCR protocols described herein.

### Example 11

### Alternative PCR Protocol

The following alternative PCR protocol can be followed utilizing the PCR mix as described in Example 5 (Table 4) in order to detect Salmonella in a sample using the primers and probes of the present invention.

Hot Start Step:

| | | |
|---|---|---|
| 1 cycle of: | 95° C. 15 min. | (Hot start) |
| | 95° C. 15 sec. | (Denaturation) |
| | 55° C. 30 sec. | (Annealing) |
| | 72° C. 30 sec. | (Extension) |

Amplification Steps:

| | | |
|---|---|---|
| 39 cycles of: | 95° C. 15 sec. | (Denaturation) |
| | 55° C. 30 sec. | (Annealing) |
| | 72° C. 30 sec. | (Extension) |

### Example 12

### Alternative PCR Protocol #2

PCR amplification was also undertaken using the conditions described in Tables 8 and 9 below. The intensity of fluorescence emitted by the fluorophore component of the molecular beacon was detected at the annealing stage of each amplification cycle. In Table 8, note that the PCR buffer contains 1.5 mM magnesium chloride (final concentration). Inclusion of additional magnesium chloride brings the final concentration to 4 mM in the reaction mixture.

TABLE 8
PCR mix.

| | Final concentration in | |
|---|---|---|
| Reagent | reconstituted reaction | |
| Qiagen PCR buffer, 10× | 1.5× | |
| Forward primer [SEQ ID NO: 32], 25 μM | 0.5 | μM |
| Reverse primer [SEQ ID NO: 33], 25 μM | 0.5 | μM |
| dNTPs, 10 mM | 0.2 | mM |
| MgCl₂, 25 mM | 4.0 | mM |
| Molecular beacon [SEQ ID NO: 34], 10 μM | 0.3 | μM |

| | |
|---|---|
| HotStarTaq, 5 U/μL | 1 U/25 μL reaction |

TABLE 9
PCR program.

| Step | Temperature | Duration | | Repeats |
|---|---|---|---|---|
| Initial polymerase activation | 95° C. | 15 | min | 1 |
| Denaturation | 94° C. | 15 | sec | 40 |
| Annealing | 55° C. | 15 | sec | |
| Elongation | 72° C. | 15 | sec | |

Fluorescence was detected in real-time using a fluorescence monitoring real-time PCR instrument, for example, a BioRad iCycler iQ™ or MJ Research Opticon™.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

1. A combination of polynucleotides for amplification and detection of a portion of a Salmonella phoP gene, said portion being less than about 500 nucleotides in length and comprising at least 60 consecutive nucleotides of the sequence set forth in SEQ ID NO:30, said combination comprising:
(a) a first polynucleotide primer comprising at least 7 nucleotides of the sequence as set forth in SEQ ID NO:1;
(b) a second polynucleotide primer comprising at least 7 nucleotides of a sequence complementary to SEQ ID NO:1; and
(c) a polynucleotide probe comprising at least 7 consecutive nucleotides of the sequence as set forth in SEQ ID NO:30, or the complement thereof.

2. The combination of polynucleotides according to claim 1, wherein said first and second polynucleotide primers comprise at least 7 nucleotides of the sequence as set forth in any one of SEQ ID NOs:16 to 22.

3. The combination of polynucleotides according to claim 1, wherein said polynucleotide probe comprises at least 7 nucleotides of the sequence as set forth in any one of SEQ ID NOs:35, 37, 39 or 41.

4. The combination of polynucleotides according to claim 1, wherein said first polynucleotide primer comprises at least 7 nucleotides of the sequence as set forth in SEQ ID NO:32 and said second polynucleotide primer comprises at least 7 nucleotides of the sequence as set forth in SEQ ID NO:33.

5. The combination of polynucleotides according to claim 1, wherein said first polynucleotide primer comprises the sequence as set forth in SEQ ID NO:32, said second polynucleotide primer comprises the sequence as set forth in SEQ ID NO:33 and said polynucleotide probe comprises the sequence as set forth in SEQ ID NO:34 or 36.

6. The combination of polynucleotides according to claim 1, wherein said first polynucleotide primer comprises the sequence as set forth in SEQ ID NO:32, said second polynucleotide primer comprises the sequence as set forth in SEQ ID NO:33 and said polynucleotide probe comprises the sequence as set forth in SEQ ID NO:38 or 40.

7. A pair of polynucleotide primers for amplification of a portion of an Salmonella phoP gene, said portion being less than about 500 nucleotides in length and comprising at least 60 consecutive nucleotides of the sequence set forth in SEQ ID NO:30, said pair of polynucleotide primers comprising:
(a) a first polynucleotide primer comprising at least 7 nucleotides of the sequence as set forth in SEQ ID NO:1; and
(b) a second polynucleotide primer comprising at least 7 nucleotides of a sequence complementary to SEQ ID NO:1.

8. The pair of polynucleotide primers according to claim 7, wherein said first and second polynucleotide primers comprise at least 7 nucleotides of the sequence as set forth in any one of SEQ ID NOs:16 to 22.

9. The pair of polynucleotide primers according to claim 7, wherein said first polynucleotide primer comprises at least 7 nucleotides of the sequence as set forth in SEQ ID NO:32 and said second polynucleotide primer comprises at least 7 nucleotides of the sequence as set forth in SEQ ID NO:33.

10. The pair of polynucleotide primers according to claim 7, wherein said first polynucleotide primer comprises the sequence as set forth in SEQ ID NO:32 and said second polynucleotide primer comprises the sequence as set forth in SEQ ID NO:33.

11. A method of detecting one or more Salmonella species in a sample, said method comprising:
(a) contacting a test sample suspected of containing, or known to contain, a Salmonella target nucleotide sequence with the combination of polynucleotides according to claim 1 under conditions that permit amplification and detection of said target sequence, and
(b) detecting any amplified target sequence,
wherein detection of an amplified target sequence indicates the presence of one or more Salmonella species in the sample.

12. The method according to claim 11, further comprising a step to enrich the microbial content of the test sample prior to step (a).

13. A kit for the detection of one or more Salmonelle species in a sample, said kit comprising:
(a) a first polynucleotide primer comprising at least 7 nucleotides of the sequence as set forth in SEQ ID NO:1;
(b) a second polynucleotide primer comprising at least 7 nucleotides of a sequence complementary to SEQ ID NO:1; and
(c) a polynucleotide probe comprising at least 7 consecutive nucleotides of the sequence as set forth in SEQ ID NO:30, or the complement thereof.

14. The kit according to claim 13, wherein said first and second polynucleotide primers comprise at least 7 nucleotides of the sequence as set forth in any one of SEQ ID NOs:16 to 22.

15. The kit according to claim 13, wherein said polynucleotide probe comprises at least 7 nucleotides of the sequence as set forth in any one of SEQ ID NOs:35, 37, 39 or 41.

16. The kit according to claim 13, wherein said first polynucleotide primer comprises at least 7 nucleotides of the sequence as set forth in SEQ ID NO:32 and said second polynucleotide primer comprises at least 7 nucleotides of the sequence as set forth in SEQ ID NO:33.

17. The kit according to claim 13, wherein said first polynucleotide primer comprises the sequence as set forth in SEQ ID NO:32, said second polynucleotide primer comprises the sequence as set forth in SEQ ID NO:33 and said polynucleotide probe comprises the sequence as set forth in SEQ ID NO:34 or 36.

18. The kit according to claim 13, wherein said first polynucleotide primer comprises the sequence as set forth in SEQ ID NO:32, said second polynucleotide primer comprises the sequence as set forth in SEQ ID NO:33 and said polynucleotide probe comprises the sequence as set forth in SEQ ID NO:38 or 40.

19. An isolated Salmonella specific polynucleotide having the sequence as set forth in SEQ ID NO:30, or the complement thereof.

20. A polynucleotide primer of between 7 and 100 nucleotides in length for the amplification of a portion of a Salmonella phoP gene, said polynucleotide comprising at least 7 consecutive nucleotides of the sequence as set forth in SEQ ID NO:30, or the complement thereof.

21. The polynucleotide primer according to claim 20, wherein said polynucleotide comprises at least 7 consecutive nucleotides of the sequence as set forth in any one of SEQ ID NOs:32, 33, 35, 37, 39 or 41.

22. The polynucleotide primer according to claim 20, wherein said polynucleotide comprises the sequence as set forth in SEQ ID NO:32 or SEQ ID NO:33.

23. A polynucleotide probe of between 7 and 100 nucleotides in length for detection of Salmonella, said polynucleotide comprising at least 7 consecutive nucleotides of the sequence as set forth in SEQ ID NO:30, or the complement thereof.

24. The polynucleotide probe according to claim 23, wherein said polynucleotide comprises at least 7 consecutive nucleotides of the sequence as set forth in any one of SEQ ID NOs:32, 33, 35, 37, 39 or 41.

25. The polynucleotide probe according to claim 23, wherein said polynucleotide comprises the sequence as set forth in any one of SEQ ID NOs:35, 37, 41 or 43.

26. The polynucleotide probe according to claim 23, wherein said polynucleotide comprises the sequence as set forth in any one of SEQ ID NOs:34, 36, 38 or 40.

27. The polynucleotide probe according to claim 23, wherein said polynucleotide further comprises a fluorophore, a quencher, or a combination thereof.
